(19) United States
(12) Patent Application Publication
(10) Pub. No.: US 2024/0139075 A1
(43) Pub. Date: May 2, 2024

(54) DENTAL MATERIAL COMPOSITIONS FOR CLEANSING, ETCHING, AND IDENTIFYING RESIDUAL CARIES & THEIR METHODS OF USE

(71) Applicant: Inter-Med, Inc., Racine, WI (US)

(72) Inventors: Alexander D. JOHNSON, Racine, WI (US); Scott LAMERAND, Racine, WI (US)

(21) Appl. No.: 18/495,093

(22) Filed: Oct. 26, 2023

Related U.S. Application Data
(60) Provisional application No. 63/381,208, filed on Oct. 27, 2022.

(51) Int. Cl. A61K 6/30; A61K 6/25; A61K 6/65; A61K 6/887; A61K 6/891

(52) U.S. Cl. CPC A61K 6/30; A61K 6/25; A61K 6/65; A61K 6/887; A61K 6/891

(57) ABSTRACT

Compositions are disclosed that simultaneously cleanse, etch, and identifying residual caries during dental restorative procedures, thereby increasing procedure efficiency. Also disclosed are methods describing the use of said compositions in treating subjects as part of dental procedures.

### CROSS-REFERENCE TO RELATED APPLICATIONS

This application claims the benefit of U.S. Provisional Application No. 63/381,208, filed Oct. 27, 2022, the entire contents of which are by this reference herein.

### BACKGROUND OF INVENTION

Dental caries, also known as tooth decay, arise from the acidic breakdown of tooth structures from bacterial activity and represent the most common noncommunicable human disease affecting more than 3.6 billion individuals worldwide. Detection of dental caries can be accomplished through several means, but primarily involves the use of dental radiographs and tactile probing with a dental explorer. Dental radiographs can be limited in their ability to detect small caries. That limitation may be exacerbated with more recently developed digital radiographs and can be prone to imaging artifacts. Some dental research has also advised against the use of dental explorers to probe potential carious lesions as the act of poking slightly demineralized dentin with a hard, metal explorer has actually been shown to promote caries formation and progression.

Regardless, once dental caries are identified, a restoration procedure ensues to replace the infected/decayed tooth structure. In the United States alone, more than 175 million caries restoration procedures are performed by dental professionals each year. This procedure typically entails mechanical removal of the affected tooth structure using various burs and tools, acid etching the area, cleaning the area, applying a bonding agent (also called an adhesive), filling the void with dental composite, curing the restoration, and final shaping and polishing to mimic the original tooth structure appearance and function. Overall, the restoration of destroyed or decayed tooth structures can be achieved through the use of various materials including dental amalgams, glass ionomer cements, composite resins, porcelain, or gold.

The goal of any restoration procedure is to remove the cause of disease and to maximize the amount of natural tooth structure remaining since no manmade material has been able to provide all the benefits (mechanical, biological, etc.) of natural tooth structure. If a dental professional does not adequately remove all the infected/decayed tooth structure, remnant bacteria will proceed to demineralize the tooth, and the restoration will inevitably fail. Such failure requires additional restoration retreatment, including the loss of additional natural tooth structure, and may necessitate endodontic therapy or tooth extraction. Not only is this an undesirable clinical outcome for the patient and dental professional, but significant time, materials, and money is wasted when restorations fail and have to be retreated, or if disease progresses. As such, it would be advantageous to be able to detect residual caries early in the restoration procedure before significant time, materials, and money are used.

In an effort to detect caries early in the restorative procedure, several products and techniques have been used. The technique most widely used by dental professionals is visible observation and tactile feel of the dentin during mechanical removal of the infected tooth structure. Since bacteria are microscopic, this technique is limited and typically results in lower disease sensitivity, or the dental professional purposefully removes more tooth structure than necessary to compensate for potential bacterial infiltration within the dentin. The CamX Spectra Caries Detection Aid (Air Techniques, Melville, NY) uses fluorescence to detect caries, but costs several thousand dollars and is limited for caries detection in fissures and on smooth surfaces, while it performs less effective for detecting interproximal caries. Various products have also been commercialized that use a visible dye to stain the infected/decayed tooth structure. The use of the CamX Spectra Caries Detection Aid, as well as other equipment-based caries detectors, and caries indication dyes all require additional products to be used within the procedure and add an extra procedural step, both of which increase cost and procedure duration.

### BRIEF DESCRIPTION OF THE DRAWINGS

FIG. 1 displays shear bond strength measurements to dentin for Formula A versus a standard protocol where a 37% phosphoric acid etch (Best Etch, Vista Apex, Racine, WI) is used in place of Formula A. Both materials were placed for 20 seconds before removal via a water rinse. As the results show, Formula A provided improved shear bond strength (>35%) compared to the conventional protocol.

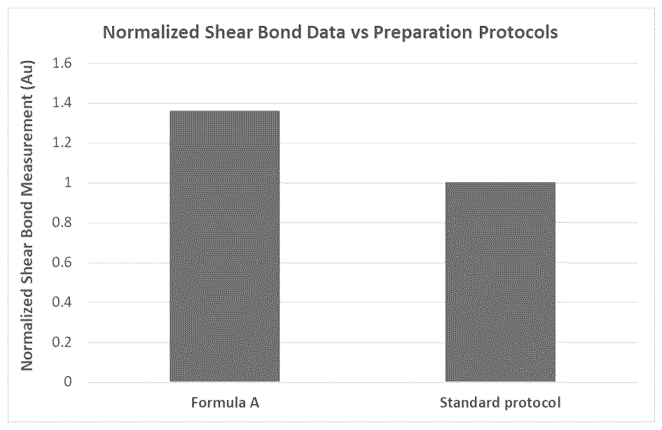

### DETAILED DESCRIPTION

The following paragraphs define in more detail the embodiments of the invention described herein. These embodiments are not meant to limit the invention or narrow the scope thereof, as it will be readily apparent to one of ordinary skill in the art that suitable modifications and adaptations may be made without departing from the scope of the invention, embodiments, or specific aspects described herein.

An object of the present invention provides, within the framework of dental restorative procedures, a composition for simultaneously cleansing, etching and identifying residual caries, which simplifies the restorative procedure. Specifically, an achievement of the compositions disclosed herein is that they ease and hasten a restorative procedure by combining several steps together, namely cleansing, etching, and residual caries identification, that were typically accomplished via separate individual products and steps. In other words, through the use of the disclosed compositions, residual caries identification can be easily combined with other steps omnipresent within restorative procedures thereby helping to facilitate early detection of residual caries prior to procedure progression. Additionally, the compositions boost the bonding strength of adhesives and other bonding agents.

In another aspect, methods of utilizing the compositions are also disclosed. In a specific embodiment, the composition comprises an aqueous solution containing a mineral acid (for etching), a caries indication dye (for identifying residual caries), and an alcohol (for cleansing). To help promote adhesion and bond strength of the restorative material to the tooth, organic monomers may also be included with the composition, for example, hydroxyethylmethacrylate (HEMA), urethane-dimethacrylate, among others. Additionally, various other components, such as thickeners, glycols, antimicrobial agents, and surfactants can be added to the composition to promote handling and cleansing.

In certain embodiments, the mineral acid is one or more of: nitric acid, phosphoric acid, hydrofluoric acid, sulfuric acid, perchloric acid, and similar acids. The mineral acids serves the function of etching tooth structures such as dentin and enamel. The alcohol is elected as one or more of: isopropyl alcohol, ethanol, butanol, pentanol, and menthol, among others.

The caries indication dye contained may function in a variety of various ways. In certain aspects, the caries indication dye may be a bacteria specific stain, for example, one or more of acid fuchsin, anilin blue, fast acid blue, fast green FCF, erythrosine, orseilline, methylene blue, among others. In other aspects, the caries indication dye may be a protein stain that binds to demineralized collagen, for example, FD&C green 3 or acid red 52, among others. The caries indication dye may be a visible or fluorescent dye. In yet other aspects, the caries indication dye may consist of a fluorescently labeled compound (e.g., antibody, protein, peptide, among others) that preferentially binds to bacteria within carious lesions. In such a situation, means to activate the dye fluorescence, as well as visualize the dye fluorescence, would be useful for clinical utility. In yet further aspects, the caries indication dye may exhibit antimicrobial properties, or may exhibit antimicrobial properties after irradiation with light of a specific wavelength (e.g., as a photosensitizer as part of photodynamic therapy).

In certain embodiments, the composition does not require shaking or mixing prior to use or application. The disclosed composition may be thixotropic. In some embodiments, the composition has a viscosity of 0.001 Pa*s−2 Pa*s. In some embodiments, the composition has a viscosity of 0.1 Pa*s−1 Pa*s. In some embodiments, the composition has a viscosity of 0.2 Pa*s−0.8 Pa*s. The composition's desirable viscosity value corresponds with it thin out or over the restoration area concomitantly providing some degree of thickness to ensure the applied layer is not too thin. In other words, the composition functions as gel-like but still easily spreads across a restoration area. To achieve the desired viscosity, the cleaning solution may contain a thickening agent (also referred to as a thickener) at a concentration of 0.5-25%. In some embodiments, the thickening agent is present at a concentration of 1-10%. In some embodiments, the thickening agent is present at a concentration of 2-5%.

Examples of suitable thixotropic agents are fumed silica and metallic silicates.

Examples of suitable thickening agents are polymers, such as one or more of polystyrene, polypropylene, polyethylene, polyacrylates, polyacrylamides, polyvinyl alcohol, fumed silica, and copolymers and surfactant combinations of the same.

The disclosed composition may also comprise additional surfactants to enhance wetting, dentinal tubule penetration, solubilization (solubility, cleaning and soil removal), debris suspension, and emulsification.

Examples of suitable wetting agents are cationic and nonionic surfactants, at an acidic pH, such as one or more of alkyldiphenyloxide disulfonates, alkyl aryl sulfonates, alkyl sulfates, alcohol ethoxylates, polyoxyethylene glycol octylphenol ethers, polyoxyethylene glycol alkylphenol ethers, polyoxyethylene glycol sorbitan alkyl esters, sorbitan alkyl esters, copolymers of polyethylene glycol and/or propylene glycol, poloxamers, sodium sterates, sodium lauryl ether sulfates, linear alkylbenzene sulfonates, benzalkonium, benzethonium, methylbenzethonium, cetylpyridinium, alkyl-dimethyl dichlorobenzene ammonium, dequalinium and phenamylinium chlorides, cetrimonium and cethexonium bromides, betaines, primary or secondary or tertiary amines, octenidine dihydrochloride, fluorosurfactants, and amine oxides.

In certain embodiments, the disclosed composition may also comprise antimicrobial agents. The function of the antimicrobial agent is to reduce or eliminate any residual microorganism present including remnant bacteria. The antimicrobial agent may optionally have affinity for dentin thereby offering substantivity after the dental procedure is complete. These antimicrobial agents may include antiseptics, disinfectants, antibiotics, antivirals, antifungals, and combinations thereof. Examples of suitable antimicrobial agents include chlorhexidine, povidone iodine, iodine, hypochlorous acid, hydrogen peroxide, amoxicillin, clindamycin, penicillin, fungizone, clotrimazole, fluconazole, miconazole, among others. In yet further aspects, the antimicrobial agent and the caries indication dye may be the same material.

Hydrotropes can also be added to surfactants for formulation stability and to reduce phase separation. Certain hydrotropes that can be used in the disclosed composition solution include, but are not limited to, sodium xylene sulfonate (SXS), ethylhexyl sulfonate (EHS), and sodium cumene sulfonate (SCS), among others.

In yet another aspect, the disclosed composition solution provides a suitable shelf life of preferably 6-48 months when stored at room temperature without displaying characteristics of chemical instability or significant decreased clinical efficacy. An example of chemical instability would be the formation of a precipitate within the composition that renders the composition less effective or useless (due to the precipitate potentially clogging various delivery or applicator tips thereby inhibiting application of the composition to the restorative site/area).

In some aspects of the present disclosure, the composition may be packaged in a bottle sized between 0.1 mL-100 mL. In some embodiments, the bottle is sized between 1 mL-10 mL. In some embodiments, the composition may be supplied in various syringes sized between 0.1 mL-100 mL. In some embodiments, the syringes are sized between 0.5 mL-5 mL. Syringe packaging is often desirable as application tips can be mated directly to the syringes of composition for immediate application to the dental restoration site/area. Conversely, the use of bottles requires an intermediate step of dispensing the composition into a well, or other vessel, and then application to the dental restoration surface using various brushes or other means. In some embodiments of the invention, it may be beneficial to have the composition stored in separate containers (e.g. two separate bottles, dual barrel syringe, etc.) and mixed by the user immediately prior to use.

The packaging of the components must be compatible for long-term storage (months to years). Satisfactory plastic resins for the packaging material may include, but are not limited to, polypropylene, polyethylene, styrene acrylonitrile, methyl methacrylate-acrylonitrile-butadiene-styrene, poly-cyclohexylenedimethylene terephthalate glycol, among others.

In some aspects, the disclosed compositions are provided as an item within a kit. The kit may include one more of the following components: application tips, application brushes, mixing tips, mixing vessels, empty syringes, an instructions for use, mixing wells or other single use vessels, a dental etchant or etchants, a dental adhesive or adhesives, a dental primer or primers, a dental composite or composites, a dental cement or cements, among other common dental products.

In some embodiments, the pH of the composition solution is acidic, meaning between 0 and 7. In some embodiments, the pH of the composition is between 0.5 and 4. In some embodiments, the pH of the composition is between 0.5 and 3. In some embodiments, the pH of the composition is between 0.5 and 1.5.

The method of using the disclosed compositions typically involves bringing the composition into contact with the mechanically prepared dental restoration site/area using an application tip or brush and letting the composition stand or “dwell” on the surface for a period of about 5 to 60 seconds. In some embodiments, the dwelling time is between about 5 to 30 seconds.

Optionally, a further step of moving the composition on the surface can follow, e.g. by distributing it over the surface (“rubbing” or “brushing”) using a suitable instrument such as a brush or application tool. The duration of the “rubbing” or “brushing” step is between about 5 to 60 seconds. In some embodiments, the duration of the rubbing or brushing step is between about 10 to 30 seconds.

Finally, the composition is removed, e.g. by rinsing with water and drying the surface with compressed air or an inert gas. At this point, if tooth structure is left stained or colored by the disclosed composition, then that would suggest remnant infected tooth structure remains and needs to be removed prior to continuing the restorative procedure. This repeated process of removing tooth structure and staining with the disclosed composition may continue until no staining of tooth structure takes place, thereby confirming no remnant bacteria and infected/decayed tooth structure is present. The described method then provides a bacteria-free foundation for bonding, cementing, or adhesively securing restorative materials via traditional dental techniques and materials, such as the use of dental primers, dental adhesives, dental cements, and/or dental composites.

In certain situations, a dental professional may not want to remove stained dentin tissue, for example, if near the dental pulp tissue. Therefore, in certain embodiments, the caries indication dye may be bleached chemically with other agents (for example, peroxides or bleaches) or photobleached using a light source.

### Definitions

For purposes of interpreting this specification, the following abbreviations, terms and definitions will apply and whenever appropriate, terms used in the singular will also include the plural and vice versa. In the event that any definition set forth below conflicts with any document incorporated herein by reference, the definition set forth below shall control.

The term “room temperature” or ambient temperature as used herein refers to common ambient temperatures ranging from about 18° C. to about 27° C.

The term “treating” refers to administering a therapy in an amount, manner, or mode effective to improve a condition, symptom, or parameter associated with a disorder. In some aspects, treating refers to the treatment of a dental ailment such as an infected tooth.

The term “substantially” as used herein means to a great or significant extent, but not completely.

As used herein, “a”, “an”, “the”, “at least one”, and “one or more” are used interchangeably.

The terms “comprises” and variations thereof do not have a limiting meaning where these terms appear in the description and claims.

The term “patient” or “subject” refers to mammals and humans. Thus, in one aspect, the subject is a mammal, or a mammal in need thereof. In one aspect, the subject is a human, or human in need thereof. In one aspect, the human or human in need thereof is a medical patient. The subject can be from about 0 years of age to 99 years of age or older.

The term “in vivo” generally means in a living subject.

The term “composition” generally refers to the chemical makeup of the disclosed multifunctional dental material composition and is synonymous with “formula.”

The term “bonding” generally refers to cementing or adhesively securing the dental restoration to various structures within a patient's mouth.

The term “restorative procedure” generally refers to the procedure used to treat dental caries and is synonymous with “restoration procedure.”

The term “tooth structure” generally refers to the material teeth are made of and comprises dentin and enamel.

The term “dental caries” generally refers to the acidic breakdown of tooth structures from bacteria and is synonymous with “tooth decay”, “dental cavity” or “dental cavities”, and “carious lesion” or “carious lesions.”

The term “substantivity” generally refers to the prolonged association between a chemical and a substrate. Within dentistry, an example of substantivity is the chemical interaction between chlorhexidine (i.e., a chemical) and dentin (i.e., a substrate) which provides prolonged antimicrobial activity even after the chlorhexidine-containing material is removed or rinsed from the dentin.

The above summary of the present invention is not intended to describe each disclosed embodiment or every implementation of the present invention. The description that follows more particularly exemplifies illustrative embodiments. In several places throughout the description, guidance is provided through lists of examples, which examples can be used in various combinations. In each instance, the recited list serves only as a representative group and should not be interpreted as an exclusive list.

### Examples

Table 1 provides a range of ingredients that may be present in compositions in the present disclosure.

TABLE 1

| | | Embodiment 1 | Embodiment 2 | |
|---|---|---|---|---|
| | | Concentration | Concentration | Embodiment 3 |
| Ingredient | Example | (w/w %) | (w/w %) | Concentration |
| Caries | FD&G Green No. 3 | 0.001-3.0% | 0.1-2.0% | 0.25-1.0% |
| indicator dye | | | | |
| Thickener | Polyvinyl alcohol | 0.0-50.0% | 1.0-20.0% | 5.0-10.0% |
| | polymer | | | |
| Alcohol | Isopropyl alcohol | 1.0-50.0% | 1.0-25.0% | 2.0-10.0% |
| Mineral acid | Nitric acid (70%) | 0.5-50.0% | 0.5-20.0% | 0.5-10.0% |
| Organic | Hydroxyethyl- | 0.0-30.0% | 1.0-25.0% | 5.0-20.0% |
| monomer | methacrylate (HEMA) | | | |
| Glycol | Propylene glycol | 0.0-75.0% | 0.0-60.0% | 0.0-50.0% |
| Antimicrobial | Chlorhexidine | 0.0-2.0% | 0.05-2.0% | 0.1-1% |
| agent | | | | |
| Water | Water | 20.0-95.0% | 40.0-90.0% | 50.0-90.0% |

Table 2 provides specific compositions consistent with the compositions in this disclosure.

TABLE 2

| | | Formula A | Formula B | Formula C |
|---|---|---|---|---|
| Ingredient | Classification | (w/w %) | (w/w %) | (w/w %) |
| Selvol 09-523 | Thickener | 7.3% | 5.5% | 5.5% |
| (polyvinyl | | | | |
| alcohol polymer) | | | | |
| Isopropyl alcohol | Alcohol | 5.0% | 3.7% | 3.7% |
| HEMA | Organic | 9.9% | 7.5% | 7.5% |
| | monomer | | | |
| Nitric Acid (70%) | Mineral acid | 5.0% | 5.0% | 5.0% |
| FD&C Green 3 | Caries | 1.0% | 0.5% | 0.5% |
| | indicator dye | | | |
| Propylene Glycol | Glycol | 0.0% | 24.3% | 24.3% |
| Water | Water | 71.8% | 53.5% | 53.05% |
| Masurf 2825 | Surfactant | 0.0% | 0.0% | 0.2% |
| Chlorhexidine | Antimicrobial | 0.0% | 0.0% | 0.25% |
| | agent | | | |

| | | | |
|---|---|---|---|
| pH | 0.9 | <1.5 | <1.5 |
| TOTAL | 100% | 100% | 100% |

Many modifications and variations of the embodiments described herein may be made without departing from the scope, as is apparent to those skilled in the art. The specific embodiments described herein are offered by way of example only.

What is claimed is:

1. A composition for etching, cleansing, and detecting caries on teeth comprising:
at least one mineral acid, and
at least one caries indication dye, and
at least one alcohol, and
water.

2. The composition of claim 1, wherein the at least one mineral acid is nitric acid.

3. The composition of claim 1, wherein the at least one caries indication dye is FD&C green 3.

4. The composition of claim 1, wherein the at least one alcohol is isopropyl alcohol.

5. The composition of claim 1, further comprising at least one thickening agent.

6. The composition of claim 1, further comprising a surfactant or blend of surfactants.

7. The composition of claim 1, further comprising a humectant or humectants.

8. The composition of claim 1, wherein the at least one mineral acid is nitric acid, wherein the at least one caries indication dye is FD&C green 3, and wherein the at least one alcohol is isopropyl alcohol.

9. The composition of claim 1, wherein the at least one caries indication dye comprises a bacteria specific stain.

10. The composition of claim 1, wherein the at least one caries indication dye comprises a protein stain that binds to demineralized collagen.

11. The composition of claim 1, further comprising at least one thickening agent that is a polyvinyl alcohol polymer.

12. The composition of claim 1, further comprising an organic monomer that is hydroxyethylmethacrylate.

13. The composition of claim 1, further comprising a glycol that is propylene glycol.

14. The composition of claim 1, further comprising one or more antimicrobial agents.

15. The composition of claim 1, further comprising an organic monomer that is hydroxyethylmethacrylate, a thickener that is a polyvinyl alcohol polymer, and wherein the at least one mineral acid is nitric acid, the at least one caries indication dye is FD&C green 3, and the at least one alcohol is isopropyl alcohol.

16. The composition of claim 1, wherein the composition pH is between 0.5 and 1.5.

17. The composition of claim 1, wherein the mineral acid is at a concentration between 0.5% and 50%.

18. The composition of claim 1, wherein the caries indicator dye is at a concentration between 0.25% and 1.0%.

19. The composition of claim 1, further comprising
an organic monomer that is hydroxyethylmethacrylate at a concentration between 5% and 10%,
a thickener that is a polyvinyl alcohol polymer at a concentration between 3% and 10%, and
wherein the at least one mineral acid is nitric acid at a concentration between 3% and 10%, the at least one caries indication dye is FD&C green 3 at a concentration between 0.1% and 1%, and the at least one alcohol is isopropyl alcohol at a concentration between 1% and 10%.

20. A method of treating dental caries, comprising:
mechanical removal of infected tooth structure from a site using dental burs;
applying a caries indication dye to the site to confirm all infected tooth structure has been removed;
acid etching the site;
cleaning the site; and
applying a bonding agent to the site;
wherein, a single composition is used to apply a caries indication dye, acid etch, and clean the site.
